(19) United States
(12) Patent Application Publication
(10) Pub. No.: US 2023/0097263 A1
(43) Pub. Date: Mar. 30, 2023

(54) METHOD AND SYSTEM TO MANAGE AND REDUCE RECALL FREQUENCY OF DISTURBING DREAMS

(71) Applicant: KONINKLIJKE PHILIPS N.V., EINDHOVEN (NL)

(72) Inventors: James Scott GARSTECK, SCOTTDALE, PA (US); Sara E. KRON, REEDSVILLE, PA (US); Dirk Ernest VON HOLLEN, CLARK, NJ (US); Benjamin Irwin SHELLY, PITTSBURGH, PA (US); Tsvetomira Kirova TSONEVA, EINDHOVEN (NL)

(21) Appl. No.: 17/955,594

(22) Filed: Sep. 29, 2022

Related U.S. Application Data
(60) Provisional application No. 63/249,745, filed on Sep. 29, 2021.

(51) Int. Cl. A61B 5/00; A61B 5/374

(52) U.S. Cl. CPC A61B 5/4812; A61B 5/374; A61M 2021/0005

(57) ABSTRACT

A method of managing and reducing the recall frequency of disturbing dreams comprises monitoring (100; 200; 300) electroencephalography (EEG) activity of a subject, detecting (102; 206; 302) an indicator of a disturbing dream of the subject based on power in a theta or alpha band of the EEG activity of the subject, and providing stimulation (104; 212; 308) to the subject at a frequency lower than the theta band in response to detecting the indicator of the disturbing dream.

### BACKGROUND OF THE INVENTION

### 1. Field of the Invention

The disclosed concept generally relates to systems and methods for reducing the recall frequency of disturbing dreams.

### 2. Description of the Related Art

Nightmares may involve images, feelings or thoughts of physical aggression, interpersonal conflict, failure/helplessness, and emotions like fear, anxiety, anger, sadness, and disgust. Nightmares are a common disturbance of rapid eye movement (REM) sleep, with the lifetime prevalence of clinically significant nightmares (at least one nightmare per week) estimated to be from 1% to 8% of the population. Nightmares are an important clinical concern, having been linked to poor sleep quality and shown to be comorbid with several sleep disorders, including insomnia. Nightmares may be idiopathic but are more prevalent in psychiatric populations and have been linked to psychopathologies such as anxiety, depression, schizophrenia, borderline personality disorder, suicide, and posttraumatic stress disorder (PTSD) as well as numerous physical ailments. Nightmares are also an important risk factor for several of these illnesses, most notably suicide and PTSD. Traumatic nightmares can be so impactful that restful sleep is negatively affected and fragmented, possibly at times rendering people unable to function normally in their daily lives.

Dream recall is associated with electrophysiological mechanisms of the brain. When used to assess the neurophysiological characteristics of individuals who frequently recall nightmares, quantitative electroencephalography (EEG) has repeatedly revealed that nightmare recallers and matched controls differ in relative spectral power, particularly in the theta band, which is an EEG oscillation associated with fear memory consolidation in the hippocampus, amygdala, and prefrontal cortex. There is higher slow-theta (2-5 Hz) for nightmare recallers than for controls during wake, non-REM sleep and REM sleep, suggesting the possible role of the slow theta frequency band in nightmare pathology. The detection of oscillatory activity reveals that both older and younger individuals recall their dream experience when the last segment of REM sleep is characterized by frontal theta oscillations. After morning awakening from REM sleep, successful dream recall was associated with higher frontal theta (5-7 Hz) activity in the EEG spectrum of subjects. Additionally, high alpha (10-14.5 Hz) power in REM sleep in the EEG spectra of nightmares.

Using neurofeedback, electrophysiological activity in the brain can be altered. For example, it has been shown that electrophysiological slow wave activity of sleep can be enhanced by providing magnetic or audio feedback in the delta frequency range.

Some have attempted to solve for bizarre dreaming and traumatic nightmares, generally involving complex solutions in clinical settings using prolonged exposure or similar treatments. Drawbacks to these solutions include cost, complexity, side effects, and inability to eliminate negative effects of nightmares.

There remains room for improvement in systems and methods of managing and reduce the recall frequency of disturbing dreams,

### SUMMARY OF THE INVENTION

Accordingly, it is an object of the disclosed concept to detect an indicator of a disturbing dream based on theta and/or alpha power in an EEG subject and to provide stimulation in response to the detection at a frequency lower than the theta band.

As one aspect of the disclosed concept, a method of managing and reducing the recall frequency of disturbing dreams comprises: monitoring electroencephalography (EEG) activity of a subject; detecting an indicator of a disturbing dream of the subject based on power in a theta or alpha band of the EEG activity of the subject; and providing stimulation to the subject at a frequency lower than the theta band in response to detecting the indicator of the disturbing dream.

As one aspect of the disclosed concept, a system for managing and reducing the recall frequency of disturbing dreams comprises: an electroencephalography (EEG) sensor structured to monitor EEG activity of a subject; a stimulation device structured to provide stimulation to the subject; and a processing unit communicatively connected to the EEG sensor and the stimulation device, the processing unit including a processor structured to: detect an indicator of a disturbing dream of the subject based on power in a theta or alpha band of the EEG activity of the subject monitored by the EEG sensor; and cause the stimulation device to provide stimulation to the subject at a frequency lower than the theta band.

As one aspect of the disclosed concept, a non-transitory computer readable medium storing one or more programs, including instructions, which when executed by a computer, causes the computer to perform a method of managing and reducing the recall frequency of disturbing dreams. The method comprises: monitoring electroencephalography (EEG) activity of a subject; detecting an indicator of a disturbing dream of the subject based on power in a theta or alpha band of the EEG activity of the subject; and providing stimulation to the subject at a frequency lower than the theta band in response to detecting the indicator of the disturbing dream.

These and other objects, features, and characteristics of the disclosed concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system to manage and reduce recall of disturbing dreams in accordance with an example embodiment of the disclosed concept;

FIG. 2 is a flowchart of a method to manage and reduce recall of disturbing dreams in accordance with an example embodiment of the disclosed concept;

FIG. 3 is a flowchart of a method to manage and reduce recall of disturbing dreams in accordance with an example embodiment of the disclosed concept;

FIG. 4 is a flowchart of a method to manage and reduce recall of disturbing dreams in accordance with an example embodiment of the disclosed concept;

FIG. 5 is a schematic diagram of a system for detecting a sleep stage in accordance with an example embodiment of the disclosed concept; and

FIG. 6 is a flowchart of a method to manage and reduce recall of disturbing dreams that combines aspects of example embodiments of the disclosed concept.

### DETAILED DESCRIPTION OF EXEMPLARY EMBODIMENTS

As required, detailed embodiments of the disclosed concept are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the disclosed concept in virtually any appropriately detailed structure.

As used herein, the singular form of “a”, “an”, and “the” include plural references unless the context clearly dictates otherwise.

As used herein, the alpha band refers to 8-12 Hz, the beta band refers to 15-30 Hz, the theta band refers to 4-8 Hz, and the delta band refers to 0.5-2 Hz.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Dream recall is favored by a higher EEG power in the theta band (4 to 8 Hz) and alpha band (8 to 12 Hz). Nightmares, in particular, are most likely to occur when theta and alpha band powers are high. In some example embodiments, in response to detection of indicators of nightmares or other disturbing dreams, sensory stimulation such as, without limitation, auditory, somatosensory, or visual stimulation may be provided to modify theta and alpha band power and/or trigger a change in sleep state such as, without limitation, REM to N2 sleep without waking. In some example embodiments, the stimulation may be provided at a frequency in a band below the theta band. For example and without limitation, the sensory stimulation may be provided in the delta band (0.5 to 2 Hz). In some example embodiments, the sensory stimulation may be provided periodically.

The intensity of the stimulation may be modulated in an open or closed-loop manner to attenuate the power in theta while preventing unnecessary sleep disturbance. In some example embodiments, the theta power is calculated as a running average of the square of the signal that results from filtering the EEG in the theta band (instantaneous theta power). In some example embodiments, potential sleep disturbances may be detected in the EEG by comparing the instantaneous alpha (8-12 Hz) and beta (15-30 Hz) powers to pre-established micro-arousal thresholds.

In some example embodiments, the effect of the stimulation is constantly monitored using the instantaneous theta power. If during a period of REM sleep with increased autonomic activation, theta power has not decreased in the latest period of time with respect to the previous period of time, then the intensity of the stimulation may be increased by a factor that is proportional to the theta power difference between the periods of time. If the theta power has decreased between the periods of time, then the stimulation intensity may be kept constant or stopped. In some example embodiments, the initial stimulation intensity when entering a period of REM sleep is set below a perceivable sensory threshold. For example, with auditory stimulation, the initial volume may be set to 20 dB-SPL. For other types of stimulation, relevant characteristics may be set below threshold levels. The stimulation may continue if alpha/beta power are below the arousal threshold.

By detecting disturbing dreams and providing stimulation to theta and alpha band power and/or trigger a change in sleep state, the recall frequency of disturbing dreams may be managed and reduced. Described hereinafter are some non-limiting example embodiments of systems and methods to manage and reduce the recall frequency of disturbing dreams.

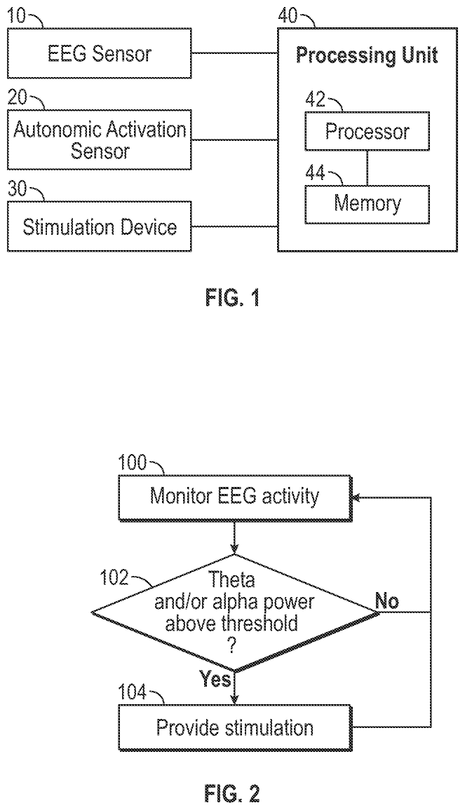

FIG. 1 is a schematic diagram of a system to manage and reduce recall of disturbing dreams in accordance with an example embodiment of the disclosed concept. In an example embodiment, the system includes an EEG sensor 10, an autonomic activation sensor 20, a stimulation device 30, and a processing unit 40 including a processor 42 and a memory 44.

The EEG sensor 10 is structured to monitor EEG brain waves. The EEG sensor 10 may comprise one or more electrodes. In some example embodiments, the EEG sensor 10 may be included in headgear such as a headband.

The autonomic activation sensor 20 is structured to detect autonomic activity. Autonomic activity may include, without limitation, electrodermal activity, heart rate, heart rate variability, respiratory rate, actigraphy, etc. The autonomic activation sensor 20 may include one or more sensors suitable for detecting corresponding autonomic activity. In some example embodiments, the autonomic activation sensor 20 may be omitted.

The stimulation device 30 is structured to provide stimulation to a subject. The stimulation may be, without limitation, auditory, somatosensory, or visual stimulation. The stimulation device 30 may include one or more components structured to provide one or more corresponding types of stimulation. For example, the stimulation device 30 may include one or more speakers, or other mechanisms, to provide auditory stimulation, one or more lights or displays to provide visual stimulation, and/or one or more device to provide somatosensory stimulation. In some example embodiments, the EEG sensor 10 and the stimulation device 30 may be integrated into the same headgear. However, it will be appreciated that they may also be provided separately.

The processing device 40 is structured to receive outputs from the EEG sensor 10 and/or autonomic activation sensor 20 and to control the stimulation device 30. The processing device 40 may include a processor 42 and a memory 44. The processor 42 may be, for example and without limitation, a microprocessor, a microcontroller, or some other suitable processing device or circuitry. The memory 44 may be any of one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory. In some embodiments of the disclosed concept, one or more routines that may be executed by the processor 42 may be stored in the memory 44 of the processing unit 40. In some example embodiments, the processing unit 40 may be integrated into the same headgear as the EEG sensor 10 and/or stimulation device 30. However, it will be appreciated that they may be provided separately.

The processor 42 is structured to execute one or more routines stored in the memory 44 to detect indicators of disturbing dreams based on outputs of the EEG sensor 10 and/or autonomic activation sensor 20 and, in response to detecting an indicator of disturbing dreams, to control the stimulation device 30 to provide stimulation to manage and reduce the recall frequency of the disturbing dreams. Some example embodiments of such routines will be described with respect to FIGS. 2-4.

FIG. 2 is a flowchart of a method to manage and reduce recall of disturbing dreams in accordance with an example embodiment of the disclosed concept. In some example embodiments, one or more steps of the method may be stored as a routine in the memory 44 executable by the processor 42.

The method begins at 100 where EEG activity of a subject is monitored. In some example embodiments, the EEG activity may be monitored by the EEG sensor 10. The EEG activity may be monitored in multiple bands including, without limitation, alpha, beta, and theta bands.

At 102, it is determined whether an indicator of a disturbing dream is present in the monitored EEG activity. In some example embodiments, the indicator of a disturbing dream is based on power in the theta band. For example, the power in the theta band may be determined by filtering the monitored EEG activity in the theta band and determining the running average of the square of the filtered signal. The resultant value may be referred to as the instantaneous theta power. The instantaneous theta power may be compared to a predetermined threshold value. If the instantaneous theta power is above a threshold value, it may be considered an indicator of a disturbing dream. It will be appreciated that the threshold value may be set in any suitable manner. In some example embodiments, the threshold value may be preset, such as by a user or medical provider. In some example embodiments, the threshold value may be determined based on user feedback. For example, a user may provide feedback on the prior night's sleep and such feedback may be used to determine the threshold value. In some example embodiments, a training period (e.g., without limitation, one week) may be used to determine the threshold value. For example, theta power, autonomic activation, and sleep disruption may be monitored during the training period and the theta power may be mapped to a probability of increased dream recall and awakening from REM sleep. The training period may be used to determine an initial threshold value. In some example embodiments, after the training period, the threshold value may continue to be updated based on data gathered while the system is in use. It will be appreciated that in some example embodiments, the threshold value may be personalized to the monitored subject. While some examples of ways to determine the threshold value have been described, it will be appreciated that any suitable manner of determining the threshold value may be used without departing from the scope of the disclosed concept.

In some example embodiments, the indicator of a disturbing dream is based on power in the alpha band. For example, the power in the alpha band may be determined by filtering the monitored EEG activity in the alpha band and determining the running average of the filtered signal. The resultant value may be referred to as the instantaneous alpha power. The instantaneous alpha power may be compared to a predetermined threshold value. If the instantaneous alpha power is above the threshold value, it may be considered an indicator of a disturbing dream. It will be appreciated that any suitable method of the determining the threshold value corresponding to alpha power may be employed, such as, without limitation, any of the methods described above corresponding to determining the threshold value corresponding to theta power.

In some example embodiments, one of the instantaneous theta or alpha power may be used to detect a disturbing dream. In some example embodiments, both the instantaneous theta and alpha power may be used to detect a disturbing dream. Furthermore, in some example embodiments, rather than instantaneous theta and/or alpha power, other methods of calculating theta and/or alpha power may be used. For example, in some example embodiments, trended or filtered theta and/or alpha power, rather than instantaneous theta and/or alpha power, may be compared to threshold values to determine if an indicator of a disturbing dream is present.

If an indicator of a disturbing dream is not detected, the method returns to 100 and EEG activity is continued to be monitored. If an indicator of a disturbing dream is detected, the method proceed to 104 where stimulation is provided. The stimulation may be provided, for example, by the stimulation device 30. The stimulation may be, for example and without limitation, auditory, somatosensory, or visual. In some example embodiments, the stimulation is provided in a band lower than the alpha or theta bands. In some example embodiments, the stimulation is provided in the delta band. For example, the stimulation may be auditory or magnetic stimulation provided at 0.5 Hz. However, the disclosed concept is not limited thereto. Stimulation in the delta band can be effective in reducing theta and/or alpha power. Furthermore, such stimulation may not result in arousal of the subject.

The stimulation may continue for a predetermined period of time in some example embodiments. In some example embodiments, the stimulation may continue until the indicator of the disturbing dream has ended (e.g., the instantaneous theta and/or alpha power has fallen below a threshold). In some example embodiments, the stimulation may be adapted based on changes in instantaneous theta and/or alpha power.

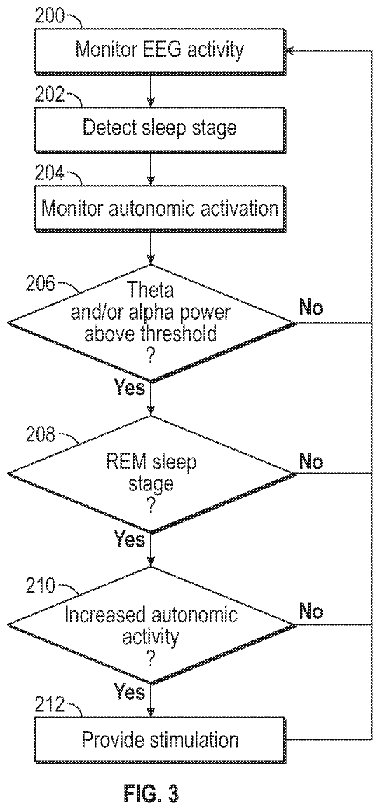

FIG. 3 is a flowchart of a method to manage and reduce recall of disturbing dreams in accordance with another example embodiment of the disclosed concept. In some example embodiments, one or more steps of the method may be stored as a routine in the memory 44 executable by the processor 42.

The method of FIG. 3 is similar to the method of FIG. 2. However, in the example embodiment described with respect to FIG. 3, additional criteria may be used to trigger stimulation. The method begins at 200 with monitoring EEG activity of a subject. The monitoring may be performed similarly to that described with respect to FIG. 2.

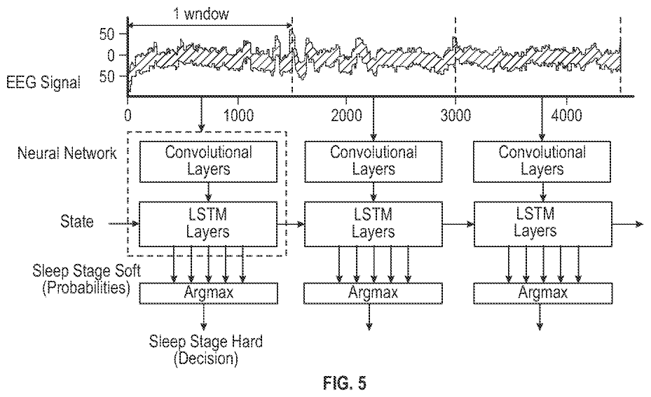

The method proceeds to 202 where a sleep stage of the subject is determined. In some example embodiments, the sleep stage is detected based on the monitored EEG activity. In some example embodiments, a deep neural network may be used to detect the sleep stage. FIG. 5 is a schematic diagram of a system that may be used to detect a sleep stage. The system is a deep neural network composed by a convolution layer (CNN) and a long-short term (LSTM) layer. The monitored EEG activity is segmented into windows that are processed by the deep neural network which outputs a set of probabilities for each sleep stage associated with each window. The stage with the largest probability is usually assigned to the window.

At 204, autonomic activity is monitored. In some example embodiments, the autonomic activation sensor 20 may be used to monitor autonomic activity. Autonomic activity may include, without limitation, electrodermal activity, heart rate, and respiratory rate. However, other autonomic activity may also be monitored.

At 206, it is determined whether there is an indicator of a disturbing dream based on instantaneous theta and/or alpha power in the EEG activity. The indicator of a disturbing dream may be detected similar to that described with respect to FIG. 2. If the indicator is not detected, the method may return to 200.

If the indicator of the disturbing dream is detected, the method proceeds to 208, where it is determined whether the subject is in the REM sleep stage. If the subject is not in the REM sleep stage, the method returns to 200. If the subject is in the REM sleep stage, the method proceeds to 210.

At 210, it is determined whether there is increased autonomic activity. To determine increased autonomic activity, the monitored autonomic activity may be compared to threshold levels, and, if the monitored autonomic activity is above a corresponding threshold level, increased autonomic activity is detected. If increased autonomic activity is not detected, the method returns to 200. If increase autonomic activity is detected, the method proceeds to 212 where stimulation is provided to the subject. The stimulation may be provided similar to that described in FIG. 2.

In an example embodiment, the stimulation is provided when the indicator of a disturbing dream is detected based on instantaneous theta and/or alpha power in the EEG activity, the subject is in the REM sleep stage, and increase autonomic activity is detected. However, it will be appreciated that one or more of the criteria may be omitted. For example, in an example embodiment, detecting increased autonomic activity may be omitted and the stimulation may be provided when the indicator of a disturbing dream is detected based on instantaneous theta and/or alpha power in the EEG activity and the subject is in the REM sleep stage. Similarly, in an example embodiment, the stimulation may be provided when he indicator of a disturbing dream is detected based on instantaneous theta and/or alpha power in the EEG activity and increase autonomic activity is detected. It will also be appreciated the detection of the indicator of the disturbing dream, the detection of the sleep stage, and the detection of increased autonomic activity may be performed sequentially, in any order, or simultaneously.

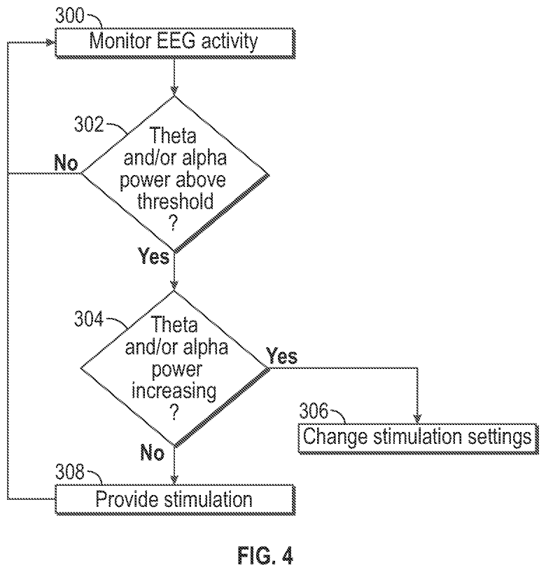

FIG. 4 is a flowchart of a method to manage and reduce recall of disturbing dreams in accordance with another example embodiment of the disclosed concept. In some example embodiments, one or more steps of the method may be stored as a routine in the memory 44 executable by the processor 42.

The method of FIG. 4 is similar to the method of FIG. 2. However, in the example embodiment shown in FIG. 4, the intensity of the stimulation is adapted. The method begins at 300 with monitoring EEG activity of the subject. The EEG activity may be monitored similar to that described with respect to FIG. 2. At 302, it is determined whether an indicator of a disturbing dream is present based on instantaneous theta and/or alpha power in the EEG activity. The detection whether the indicator of a disturbing dream is present may be done similar to that described with respect to FIG. 2. If the indicator of a disturbing dream is not detected, the method returns to 300. If the indicator of a disturbing dream is detected, the method proceeds to 304.

At 304, it is determined whether the theta and/or alpha power in the EEG activity is increasing. Increasing power may be detected by comparing the theta and/or alpha power currently detected to the theta and/or alpha power previously detected. For example, the theta and/or alpha power detected in a predetermined window of time may be compared to the theta and/or alpha power in the previous window of time. If the theta and/or alpha power is not increasing, the method may proceed to 308 where stimulation is provided at the same intensity as was previously used.

If, at 304, the theta and/or alpha power is determined to be increasing, the method proceeds to 306. At 306, the settings of the stimulation are changed, for example, by increasing the intensity of the stimulation. In some example embodiments, the intensity of the stimulation is increased by a factor that is proportional to the increase in the theta and/or alpha power. For example, if the theta and/or alpha power has increase by 10%, then the intensity of the stimulation may be increased by 2% (i.e., a factor of ⅕). However, it will be appreciated that this factor is just an example and other factors may be employed without departing from the scope of the disclosed concept.

In some example embodiments, sleep micro-arousals may be detected based on alpha or beta power. For example, if alpha or beta power (e.g., calculated by root-mean-square) of the EEG activity exceeds preset levels for a predetermined period of time (e.g. at least 500 ms), then a sleep micro-arousal is detected. The detection of the sleep micro-arousal may cause stimulation to be temporarily stopped. In some cases the increasing intensity of stimulation in response to increasing theta and/or alpha power may cause a micro-arousal that will disrupt the disturbing dream in the case that the stimulation was not effective in reducing the theta and/or alpha power. In some example embodiments, the intensity of stimulation may be increase to intentionally cause a micro-arousal to disrupt the disturbing dream. For example, after a predetermined period of stimulation, the intensity of the stimulation may be increased to a level to cause a micro-arousal to disrupt the disturbing dream. As another example, when the theta and/or alpha power reaches a threshold level, the intensity of stimulation may be increased to a level to cause a micro-arousal to disrupt the disturbing dream. A micro-arousal can be beneficial to disrupt a disturbing dream without waking the subject. For example, the subject may just temporarily change sleep stages, but remain asleep from the micro-arousal.

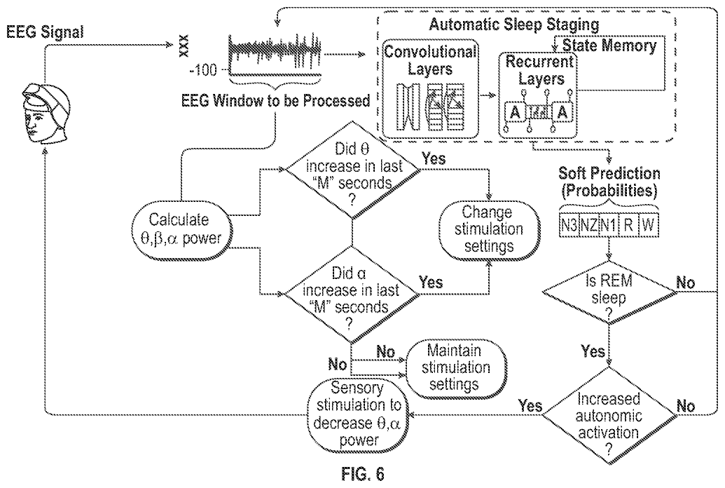

While some example embodiments have been described, it will be appreciated that aspects of the various embodiments may be modified or combined without departing from the scope of the disclosed concept. For example, aspects of additional criteria for initiating stimulation, as described with respect to FIG. 3 may be combined with aspects of adapting stimulation, as described with respect to FIG. 4. For example, a method including aspects of all embodiments may be used without departing from the scope of the disclosed concept. As an example, FIG. 6 is a flowchart of a method that combines aspects of the embodiments described in FIGS. 3 and 4.

The disclosed concept can also be embodied as computer readable codes on a tangible, non-transitory computer readable recording medium. The computer readable recording medium is any data storage device that can store data which can be thereafter read by a computer system. Non-limiting examples of the computer readable recording medium include read-only memory (ROM), non-volatile random-access memory (RAM), CD-ROMs, magnetic tapes, floppy disks, disk storage devices, and optical data storage devices.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A method of managing and reducing the recall frequency of disturbing dreams, the method comprising:
monitoring electroencephalography (EEG) activity of a subject;
detecting an indicator of a disturbing dream of the subject based on power in a theta or alpha band of the EEG activity of the subject; and
providing stimulation to the subject at a frequency lower than the theta band in response to detecting the indicator of the disturbing dream.

2. The method of claim 1, wherein the indicator of the disturbing dream is detected by comparing power in the theta or alpha band of the EEG activity of the subject to a threshold value.

3. The method of claim 1, wherein the indicator of the disturbing dream is detected by calculating an instantaneous power of the theta or alpha band of the EEG activity of the subject as a running average of a square of the theta or alpha band of the EEG of the subject and comparing the instantaneous power of the theta or alpha band of the EEG of the subject to a threshold value.

4. The method of claim 1, further comprising:
detecting a sleep stage of the subject,
wherein providing the stimulation to the subject is in response to detecting the indicator of the disturbing dream and detecting the sleep stage of the subject is a rapid eye movement sleep stage.

5. The method of claim 4, wherein detecting the sleep stage of the subject includes using a deep neural network composed by a convolution layer and a long-short term memory layer.

6. The method of claim 1, further comprising:
detecting autonomic activity of the subject,
wherein providing the stimulation to the subject is in response to detecting the indicator of the disturbing dream and detecting that autonomic activity of the subject is increasing.

7. The method of claim 6, wherein the autonomic activity includes at least one of electrodermal activity, heart rate, heart rate variability, and respiratory rate.

8. The method of claim 1, further comprising:
determining a change in power in the theta or alpha band of the EEG activity of the subject; and
in response to determining an increase in power in the theta or alpha band of the EEG activity of the subject, increasing an intensity of the stimulation by a factor proportional to the increase in power in the theta or alpha band of the EEG activity of the subject.

9. The method of claim 1, further comprising:
detecting a micro-arousal of the subject based on power in the alpha or a beta band of the EEG activity of the subject; and
stopping the stimulation in response to detecting the micro-arousal.

10. The method of claim 9, wherein detecting the micro-arousal is based on an increase in power in the alpha or beta band of the EEG activity of the subject for a predetermined period of time.

11. The method of claim 1, further comprising:
increasing an intensity of the stimulation to cause a micro-arousal of the subject.

12. The method of claim 1, wherein the stimulation includes at least one of auditory, somatosensory, and visual stimulation.

13. The method of claim 1, wherein the frequency of stimulation is in the delta band.

14. A system for managing and reducing the recall frequency of disturbing dreams, the system comprising:
an electroencephalography (EEG) sensor structured to monitor EEG activity of a subject;
a stimulation device structured to provide stimulation to the subject; and
a processing unit communicatively connected to the EEG sensor and the stimulation device, the processing unit including a processor structured to:
detect an indicator of a disturbing dream of the subject based on power in a theta or alpha band of the EEG activity of the subject monitored by the EEG sensor; and
cause the stimulation device to provide stimulation to the subject at a frequency lower than the theta band.

15. A non-transitory computer readable medium storing one or more programs, including instructions, which when executed by a computer, causes the computer to perform a method of managing and reducing the recall frequency of disturbing dreams, the method comprising:
monitoring electroencephalography (EEG) activity of a subject;
detecting an indicator of a disturbing dream of the subject based on power in a theta or alpha band of the EEG activity of the subject; and
providing stimulation to the subject at a frequency lower than the theta band in response to detecting the indicator of the disturbing dream.
